(19) United States
(12) Patent Application Publication
(10) Pub. No.: US 2006/0008831 A1
(43) Pub. Date: Jan. 12, 2006

(54) Methods and systems for predicting protein-ligand coupling specificities

(76) Inventors: Kodangattil R. Sreekumar, Plainsboro, NJ (US); Youping Huang, Plainsboro, NJ (US); Mark H. Pausch, Rocky Hill, NJ (US); Kamalakar Gulukota, Hyderabad (IN)

Correspondence Address: NIXON PEABODY LP, 401 9TH STREET, N.W., SUITE 900, WASHINGTON, DC, 20004, US

(21) Appl. No.: 11/176,621

(22) Filed: Jul. 8, 2005

Related U.S. Application Data
(60) Provisional application No. 60/586,409, filed on Jul. 9, 2004.

(51) Int. Cl. C12Q 1/68; G01N 33/53; G06F 19/00

(52) U.S. Cl. 435/6; 435/7.1; 702/19

(57) ABSTRACT

The invention provides methods and systems for predicting or evaluating protein-ligand coupling specificities. A pattern recognition model can be trained by selected sequence segments of training proteins which have a specified ligand coupling specificity. Each selected sequence segment is believed to include amino acid residue(s) that may contribute to the ligand coupling specificity of the corresponding training protein. Sequence segments in a protein of interest can be similarly selected and used to query the trained model to determine if the protein of interest has the same ligand coupling specificity as the training proteins. In one embodiment, the pattern recognition model employed is a hidden Markov model which is trained by concatenated cytosolic domains of GPCRs which have interaction preference to a specified class of G proteins. This trained model can be used to evaluate G protein coupling specificity of orphan GPCRs.

This application claims priority to U.S. Provisional Application No. 60/586,409, filed Jul. 9, 2004, the entire content of which is incorporated herein by reference.

### TECHNICAL FIELD

The invention relates to methods and systems for predicting GPCR-G protein and other protein-ligand coupling specificities.

### BACKGROUND

G protein-coupled receptors (GPCRs) comprise a super family of cell surface receptors which mediate the majority of transmembrane signal transduction in living cells. A variety of physiological functions are regulated by GPCRs, for example, neurotransmission, visual perception, smell, taste, growth, secretion, metabolism, and immune responses. Agonists and antagonists of GPCRs and agents that interfere with cellular pathways regulated by GPCRs are widely used drugs. Drug targeting of GPCRs is aimed at treating conditions including, but not limited to, osteoporosis, endometriosis, cancer, retinitis pigmentosa, hyperfunctioning thyroid adenomas, precocious puberty, x-linked nephrogenic diabetes, hyperparathyroidism, hypocalciuric hypercalcaemia, short-limbed dwarfism, obesity, glucocorticoid deficiency, diabetes, and hypertension.

A structural feature common to GPCRs is the presence of seven transmembrane-spanning α-helical segments connected by alternating intracellular (i1, i2, and i3) and extracellular (o2, o3, and o4) loops, with the amino terminus (o1) located on the extracellular side and the carboxy terminus (i4) on the intracellular side. GPCRs bind to ligands through the extracellular or transmembrane domains. Ligand binding is believed to result in conformational changes of GPCRs that lead to a cascade of intracellular events mediated by effector proteins. The path of the intracellular cascade is determined by the specific class of G proteins with which the receptors interact. The heterotrimeric G proteins, composed of α, β, and γ subunits, are classified based on the α subunit. The α subunit belongs to one of the four classes: (1) Gₛ, which stimulates adenylyl cyclase (e.g., Gₛ and G_{olf}); (2) G_{i/o}, which inhibits adenylyl cyclase and regulates ion channels (e.g., Gᵢ₁, Gᵢ₂, Gᵢ₃, Gₒ₁, Gₒ₂, Gₒ₃, G_{z}, Gₜ₁, Gₜ₂, and G_{gust}); (3) G_{q/11}, which activates phospholipase C β (e.g., G_{q}, G₁₁, G₁₄, and G_{15/16}); and (4) G_{12/13}, which activates the Na⁺/H⁺ exchanger pathway (e.g., G₁₂ and G₁₃).

At least five different G protein β subunits and eleven γ subunits have been identified. G protein βγ complexes are relatively stable and, therefore, are usually regarded as one functional unit. It is believed that the main role of Gβγ in receptor coupling is not to provide a binding surface for the receptor, but rather to help keep Gα in the optimal conformation for receptor binding.

Prediction of the interaction between GPCRs and G proteins is of great interest for the discovery of drug targets but is plagued with many issues. One difficulty for discovering drug targets is that the binding modes for agonists acting on GPCRs are almost as diverse as the chemical nature of the ligands. Even agonists acting at the same receptor may not necessarily share an overlapping binding site. Many GPCRs, although preferentially linked to a certain subfamily of G proteins, can also couple to other classes of G proteins. This promiscuity makes it more difficult to understand the coupling process and decreases the specificity of potential drugs. Another issue involves multiple structural classes of GPCRs that share little or no sequence homology. Attempts to predict the G protein coupling profile of a newly cloned GPCR based simply on its primary sequence have little success, particularly if the new sequence has a low degree of sequence homology with receptors whose coupling preferences are known.

Various biochemical approaches have been developed to determine GPCR coupling specificity and to elucidate the mechanism of the molecular specificity. Despite intensive research for more than 15 years, the coupling specificity of many GPCRs has yet to be experimentally defined. Determining the coupling specificity is an essential step in understanding the biology of a GPCR and important for the development of cell-based assays used in discovering therapeutic agents. The development of methods for accurate determination of G protein coupling would be of particular use in the study of orphan GPCRs (oGPCRs), those GPCR-like sequences for which no ligand is yet known. While empirical methods exist for predicting the G protein coupling selectivity of oGPCRs, the approaches often have high error rates and are not predictive in many instances. Thus, improved methods for predicting the G protein coupling selectivity of GPCRs would be of significant utility.

### SUMMARY OF THE INVENTION

The invention provides methods and systems for evaluating GPCR-G protein and other protein-ligand coupling specificities. The invention employs knowledge-restricted pattern recognition models which are trained by selected sequence segments of training proteins. Each selected sequence segment is believed to include amino acid residue(s) that may reside at the interface of the protein-ligand interaction, or contribute to the ligand coupling specificity of the corresponding training protein. Similarly-situated sequence segments in a protein of interest can be selected and used to query a trained model. The overall fit of the query sequence to the trained model is, therefore, indicative of whether the protein of interest possesses the same ligand coupling specificity as the training proteins. Pattern recognition models suitable for the present invention include, but are not limited to, hidden Markov models (HMMs), principal component analysis, support vector machines, and partial least squares analysis.

In one aspect, the invention features methods for evaluating G protein coupling specificity of a GPCR of interest. These methods comprise:

training a pattern recognition model with a plurality of training sequences, where the training sequences are derived from a group of training GPCRs which have interaction preference to, or are capable of interacting with, a specified class of G proteins, where each training sequence comprises a concatenation of two or more non-contiguous sequence segments of a training GPCR, and each of the non-contiguous sequence segments includes an intracellular sequence of the training GPCR; and

querying the trained model with a query sequence which comprises a concatenation of two or more non-contiguous sequence segments of the GPCR of interest. Like the training sequences, each concatenated sequence segment in the query sequence also includes a GPCR intracellular sequence. Therefore, a match or no-match of the query sequence to the trained model is indicative of whether the GPCR of interest has interaction preference or is capable of interacting with the specified class of G proteins.

Sequence segments suitable for the construction of training or query sequences can be selected based on a multiple sequence alignment of the training GPCRs and the GPCR of interest. The relative positions of the extracellular, transmembrane, and intracellular sequences of these GPCRs can be determined. Similarly-situated sequence segments in the multiple sequence alignment, such as intracellular sequences or cytosolic domains, can be selected for the construction of training or query sequences. Multiple sequence alignment programs suitable for this purpose include, but are not limited to, the T-Coffee model. Transmembrane helices in GPCRs can also be predicted using TMHMM, TopPred, or other programs to facilitate the multiple sequence alignment.

In many embodiments, the non-contiguous sequence segments used for the construction of training or query sequences are cytosolic domains of GPCRs. In one example, each training and query sequence employed includes a concatenation of two or more cytosolic domains of a corresponding GPCR. In another example, each training and query sequence employed includes a concatenation of four cytosolic domains of a corresponding GPCR.

In still another example, a pattern recognition model employed in the invention is a hidden Markov model (HMM). A query against a trained HMM produces an E-value or an HMMER score which indicates a match or no-match of the query sequence to the trained model.

In a further example, the specified class of G protein that is being investigated is selected from the group consisting of G_{i/o} class, G_{q/11} class, Gₛ class, and G_{12/13} class, and the GPCR of interest is an orphan GPCR.

The invention also features methods for identifying modulators of interactions between a GPCR of interest and G proteins. These methods include:

identifying a class of G proteins capable of interacting with the GPCR of interest according to a method described herein; and

monitoring an interaction between the GPCR of interest and a G protein selected from the class in the presence or absence of an agent.

A change in the interaction in the presence of the agent, as compared to in the absence of the agent, indicates that the agent is capable of modulating the interaction between the GPCR of interest and the selected G protein.

In a non-limiting example, the agent thus identified is an agonist or antagonist of the GPCR of interest. In another non-limiting example, the GPCR of interest being investigated is an orphan GPCR.

The invention further features methods for modulating signal transduction pathways mediated by a GPCR of interest. These methods include:

identifying a class of G proteins capable of interacting with the GPCR of interest according to a method described herein;

providing an agent capable of modulating a signal transduction pathway mediated by a G protein selected from the class thus identified; and

introducing the agent into a cell which comprises the GPCR of interest and the selected G protein.

By modulating the signal transduction pathway mediated by the selected G protein, the agent can also alter activities downstream of the GPCR of interest.

The invention also features methods for building pattern recognition models for evaluating G protein coupling specificity of GPCRs. These methods include:

preparing training sequences from a plurality of GPCRs which have a specified G protein coupling specificity, where each training sequence comprises a concatenation of two or more non-contiguous sequence segments of a GPCR, and each of the non-contiguous sequence segments includes an intracellular sequence of the GPCR; and

training a pattern recognition model with the training sequences.

In one example, the pattern recognition model being built is an HMM, and each training sequence employed comprises a concatenation of four cytosolic domains of a training GPCR.

The invention further features systems suitable for the evaluation of G-protein coupling specificity of GPCRs. These systems typically include computers or work stations which comprise a pattern recognition model trained by a plurality of training sequences. Each of the training sequences comprises a concatenation of two or more non-contiguous sequence segments of a GPCR which has a specified G protein coupling specificity, and each of the non-contiguous sequence segments comprises an intracellular sequence of the GPCR. In a non-limiting example, the pattern recognition model employed is an HMM, and each training sequence comprises a concatenation of four cytosolic domains of a training GPCR.

In addition, the invention features methods for evaluating ligand coupling specificity of other proteins. These methods comprise:

training a pattern recognition model (e.g., an HMM) with a plurality of training sequences, where the training sequences are derived from a group of training proteins which have a specified ligand coupling specificity, and each of the training sequences comprises a concatenation of two or more non-contiguous sequence segments of a training protein; and

querying the trained model with a query sequence which comprises a concatenation of two or more non-contiguous sequence segments of a protein of interest.

The concatenated sequence segments in each training and query sequence are similarly situated in the original proteins (e.g., similarly situated in a multiple sequence alignment of the original proteins). Therefore, a match or no-match of the query sequence to the trained model is indicative of whether the protein of interest has the same ligand coupling specificity as the training proteins. Systems comprising a model thus trained are also contemplated by the invention.

Other features, objects, and advantages of the invention are apparent in the detailed description that follows. It should be understood, however, that the detailed description, while indicating preferred embodiments of the invention, is given by way of illustration only, not limitation. Various changes and modifications within the scope of the invention will become apparent to those skilled in the art from the detailed description.

### BRIEF DESCRIPTION OF THE DRAWINGS

The drawings are provided for illustration, not limitation.

FIG. 1 shows a data set of mean scores used in the discriminant analysis, where the I, Q, and S scores represent the G_{i/o}, G_{q/1}, and Gₛ classes, respectively.

FIG. 2A illustrates a radar plot of E-values obtained during the model building and testing process described in Example 3, where the radii of the plot correspond to the observed E-values for melanocortin 3 receptor (MC3R), with each radial axis representing one evaluation of the models. The test protein was included in the test set 33 times and hence the radial axes are numbered 1-33.

FIG. 2B depicts another radar plot of E-values obtained during the model building and testing process described in Example 3, where the radii of the plot correspond to the observed E-values for follicle stimulating hormone receptor (FSHR), with each radial axis representing one evaluation of the models. The test protein was included in the test set 26 times and hence the radial axes are numbered 1-26.

### DETAILED DESCRIPTION

The present invention features methods of using pattern recognition models to predict GPCR-G protein and other protein-ligand coupling specificities. A pattern recognition model can be trained on proteins which have a specified ligand coupling specificity. As opposed to the use of the full-length sequences, the training can be performed on selected sequence segments in each training protein. Each selected sequence segment includes amino acid residue(s) that may reside at the interface of the protein-ligand interaction, or contribute to the ligand coupling specificity of the corresponding training protein. A pattern recognition model thus trained is therefore a knowledge-restricted model. In many embodiments, the selected sequence segments in each training protein are concatenated to produce a training sequence, which is used to train and build a knowledge-restricted pattern recognition model. Similarly-situated sequence segments in a protein of interest can be selected and concatenated to produce a query sequence. The overall fit of the query sequence to the trained model is, therefore, indicative of whether the protein of interest has the same ligand coupling preference as the training proteins.

Pattern recognition models suitable for the present invention include, but are not limited to, HMMs, principal component analysis, support vector machines, and partial least squares analysis. HMMs are often used for multiple sequence alignments, but can also be used for analyzing the periodic patterns in a single sequence. See Krogh, et al., J. MOL. BIOL., 235:1501-1531 (1994); and Eddy, BIOINFORMATICS REVIEW, 14:755-763 (1998). Generally speaking, an HMM is a statistical model for an ordered sequence of symbols and acts as a stochastic state machine that generates a symbol each time a transition is made from one state to the next. Transitions between states are specified by transition probabilities. State and transition probabilities are multiplied to obtain a probability of the give sequence. The hidden aspect of an HMM is that there is no one-to-one correspondence between the states and the symbols.

One advantage of HMMs is that HMMs have a formal probabilistic basis. All the scoring parameters employed in HMMs can be set by probability theory. This probabilistic basis allows HMMs to be trained from unaligned sequences, if a trusted alignment has not been identified. As used herein, “training” refers to the process by which the parameters of a model are selected and adjusted such that the model represents the observed variations in the training sequences. For multiple sequence alignment, the training may include optimizing the transition probabilities between states and the amino acid compositions of each match state in the model until the best HMM for all of the training sequences is obtained.

Suitable programs for construction of HMMs include, but are not limited to, HMMER (Washington University School of Medicine, Saint Louis, Mo.), SAM (Jack Baskin School of Engineering, University of California, Santa Cruz, Calif.), and PFTOOLS (The ISREC Bioinformatics Group).

HMMER is an implementation of profile HMMs. See HMMER USER'S GUIDE (by Eddy, HHMI/Washington University School of Medicine, October 2003), the entire content of which is incorporated herein by reference. One application of HMMER is to identify unknown members of a protein family, where the protein family has a number of conserved residues or topologies which are separated by characteristic spacing or sequences. In one format, a multiple sequence alignment is first constructed to delineate these conserved resides or topologies. A profile HMM is then built from the multiple sequence alignment by using “hmmbuild” and optionally calibrated by “hmmcalibrate.” Calibration increases the sensitivity of database search. A sequence of interest can be queried against the HMM by using “hmmpfam.” The query produces an E value and a score for each HMM. The E-value and the score represents the confidence that the sequence of interest belongs to the protein family upon which the HMM is constructed.

The E-value is calculated from the bit score, and reflects how many false positives a query would have expected to produce at or above this bit score. For instance, an E-Value of 0.1 means that there is a 10% chance that the query would have resulted in an equally good hit in a query of an HMM built from non-related or non-homologous training sequences. Unlike the raw score, the E-value is dependent on the size of the HMM database being searched.

An HMMER score is a criterion that represents whether the query sequence is a better match to the HMM model (positive score) or to the null model of non-related or non-homologous sequences (negative score). An HMMER score of above log2 of the number of sequences in the HMM database often suggests that the query sequence is a true member or homologue of the protein family from which the HMM is derived.

Other pattern recognition models can also be used for the present invention. These models include, but are not limited to, principal component analysis, partial least squares analysis, and support vector machines. Principal component analysis is a technique for reducing the dimensionality of the data set by transforming the original variables into a set of new variables (the principal components, or PCs). See PRINCIPAL COMPONENT ANALYSIS (by Jolliffe, Springer, N.Y., 1986). PCs are uncorrelated and can be ordered such that the kth PC has the kth largest variance among all PCs. Partial least squares regression is an extension of the multiple linear regression model for constructing predictive models that can handle redundant variables. See Geladi and Kowalski, ANALYTICA CHIMICA ACTA, 185:1-17 (1986). Support vector machines (SVMs) are a supervised machine learning technique. See AN INTRODUCTION TO SUPPORT VECTOR MACHINES (by Cristianini and Shawe-Taylor, Cambridge University Press, 2000). In SVM, the original input space is mapped into a high dimensional dot product space called feature space, and the optimal hyperplace in the feature space is determined to maximize the generalization ability of the classifier. SVM based classification is often built to minimize the structural misclassification risk, leading to enhanced generalization properties.

A pattern recognition model of the present invention can be trained and built for any protein family whose members can be divided into different classes based on their respective ligand coupling specificities. Examples of these protein families include, but are not limited to, GPCRs, transcription factors, ion channels, kinases, phosphatases, and proteases. Suitable ligands for these proteins include, but are not limited to, polypeptides, lipids, polysaccharides, DNA, RNA, or other molecules that can be classified based on their activities, sequences, structures, or other physical, chemical or biological features.

To build a pattern recognition model, proteins with known ligand coupling specificities can be grouped based on their respective ligand coupling preferences. Each group of proteins having a specified ligand coupling specificity can be used as training proteins to train a pattern recognition model such that the trained model can discriminably recognize proteins with the same ligand coupling specificity.

In one aspect, sequence segments can be selected from each training protein. These segments are non-contiguous, and can be separated from each other by at least 2, 3, 4, 5, 6, 7, 8, 9, 10, 15, 20, or more residues. Each sequence segment includes amino acid residue(s) that may reside at the interface of the protein-ligand interaction or contribute to the ligand coupling specificity of the corresponding training protein. A training sequence principally composed of these selected segments can be prepared and used to train and build a pattern recognition model of the present invention.

A pattern recognition model thus constructed is a knowledge-restricted model because of the use of a priori knowledge during its construction. Sequence segments in a protein of interest can be similarly selected and used to query the trained model for the prediction of the ligand coupling specificity of the protein of interest.

In one embodiment, all but the amino acid residues in the selected sequence segments are removed from each training and query protein. The remaining segments are then concatenated to generate respective training or query sequences. In one example, each training or query sequence is prepared by concatenating the selected segments in the order as they appear in the original protein. In another example, each training and query sequence is prepared by concatenating the selected segments in an order that is different from that in the original protein. In still another example, the amino acid residues in each selected segment are rearranged in a specified manner, provided that the same arrangement is used for both the training and query sequences.

In many embodiments, the location of each selected sequence segment in a training or query protein is determined through a multiple sequence alignment of the training and query proteins. The multiple sequence alignment allows the selected sequence segments to be structurally or functionally related among different proteins. Multiple sequence alignment programs suitable for this purpose include, but are not limited to, CLUSTLAW (Thompson, et al., NUCLEIC ACIDS RES., 22:4673-4680 (1994)), CLUSTALX, (Thompson, et al., NUCLEIC ACIDS RES., 25:4876-4882 (1997)), MSA (Gupta, et al., J. COMPUT. BIOL., 2:459-472 (1995)), PRALINE (Heringa, COMPUT. CHEM., 23:341-364 (1999)), DIALIGN segment alignment (Morgenstern, et al., PROC. NATL. ACAD. SCI., 93:12098-12103 (1996)), MultAlin (Corpet, NUCLEIC ACIDS RES., 16:10881-10890 (1988)), PRRP progressive global alignment (Gotoh, J. MOL. BIOL., 264:823-838 (1996)), SAGA genetic algorithm (Notredame and Higgins, NUCLEIC ACIDS RES., 24:1515-1524 (1996)), Aligned Segment Statistical Evaluation Tool (Asset) (Neuwald and Green, J. MOL. BIOL., 239:698-712 (1994)), BLOCKS (Henikoff and Henikoff, NUCLEIC ACIDS RES., 19:6565-6572 (1991)), eMOTIF (Nevill-Manning, et al., PROC. NATL. ACD. SCI., 95:5865-5871 (1998)), and the Gibbs sampler statistical method (Lawrence, et al., SCIENCE, 262:208-214 (1993), and Liu, et al., J. AM. STAT. ASSOC., 90:1156-1170 (1995)). A multiple sequence alignment employed in the present invention can be a global alignment, a local alignment, or a combination thereof. Other types of sequence alignment algorithms can also be used for the present invention.

In a non-limiting example, T-Coffee is used to provide a multiple sequence alignment of the training and query proteins. T-Coffee is a sequence alignment model that provides a library of alignment information independent of the phylogenetic spread of the sequences in the tests (Notredame, et al., J. MOL. BIOL., 302:205-17 (2000)). The information in the library enables an analysis of all the pairs while each step of the progressive multiple alignment is carried out, thus providing both global and local pair-wise alignments for increased accuracy. The model's accuracy lies in its ability to use all the information in the library instead of only the two sequences being compared.

Programs or algorithms for predicting protein functions, structures or topologies can also be used for selecting proper segments in each training or query protein. Protein domains with distinct or conserved primary, secondary or tertiary structures can be identified by using numerous protein classification or structure prediction programs. Suitable programs for this purpose include, but are not limited to, eMOTIF (Nevill-Manning, et al., supra), DIP (Xenarios, et al., NUCLEIC ACIDS RES., 28:289-291 (2000)), HOMSTRAD (Mizuguchi, et al., PROTEIN SCI., 7:2469- (1998)), HSSP (Dodge, et al., NUCLEIC ACIDS RES., 26:313-315 (1998)); NetOGly (Hansen, et al., NUCLEIC ACIDS RES., 25:278-282 (1997)), Pfam (Sonnhammer, et al., NUCLEIC ACIDS RES., 26:320-322 (1998)), PIR (Barker, et al., METHODS ENZYMOL., 266:59-71 (1996)), PSORT (website “psort.nibb.acjp”), SMART (Schultz, et al., PROC. NATL. ACAD. SCI., 95:5857-5864 (1998)), TargetDB (Wei and O'Connell, BIOINFORMATICS, 15:765-766 (1999)), the environmental template method (Bowie, et al., METHODS ENZYMOL., 266:598-616 (1996); and Johnson, et al., METHODS ENZYMOL., 266:575-598 (1996)), the contact potential method (Sippl, J. MOL. BIOL., 213:859-883 (1990); and Alexandrov, et al., PAC. SYMP. BIOCOMPUT., 1996:53-72 (1996)), the discrete-space model (Stultz, et al., ADV. MOL. CELL BIOL., 22B:447-506 (1997); and White, et al., MATH. BIOSCI., 119:35-75 (1994)), and the nearest-neighbor method (Salamov and Solovyev, J. MOL. BIOL., 247:11-15 (1997); and Frishman and Argos, PROTEINS, 27:329-335 (1997)). The Conserved Domain Database and Search Service provided by National Center for Biotechnology Information (NCBI) (Bethesda, Md.) can also be used. The Conserved Domain Database includes domains derived from SMART and Pfam, as well as contributions from other sources, such as COG (Tatusov, et al., SCIENCE, 278:631-637 (1997)). The Conserved Domain search employs the reverse position-specific BLAST algorithm, in which the query sequence is compared to a position-specific score matrix prepared from the underlying conserved domain alignment.

In one embodiment, TMHMM (Krogh, et al., J. MOL. BIOL., 305:567-580 (2001)) is employed for predicting the membrane topology of a training or query protein. TMHMM is a protein topology prediction method based on HMM. The method incorporates hydrophobicity, charge bias, helix lengths, and grammatical constraints into an HMM model.

In another embodiment, TopPred is used to predict transmembrane helices missed by TMHMM. TopPred is a program designed to predict the topologies of eukaryotic and prokaryotic proteins (Claros and Heijne, COMPUT. APPL. BIOSCI., 10:685-686 (1994)). Hydrophobicity profiles and transmembrane segments can also be calculated from the program. For eukaryotic proteins, there are three criteria for determining the topology of a transmembrane protein: (1) the difference in positively charged residues between the two sides of the membrane; (2) the net charge difference between the 15 N-terminal and C-terminal residues flanking the most N-terminal transmembrane segment; and (3) the overall amino acid composition of loops longer than 60 residues analyzed by the compositional distance method.

In many examples, the present invention features pattern recognition models capable of predicting G protein coupling specificity of GPCRs. Experimental evidence indicates that the intracellular loops and the carboxy-terminal end of GPCRs are involved in G protein coupling, and the cytoplasmic ends of the transmembrane helices also contribute towards G-protein recognition and activation. A pattern recognition model with an exhaustive enumeration of all possible combinations of the four cytosolic domains will likely give rise to too many variables. Such a model may also be narrowly trained and therefore have limited ability to generalize.

By concatenating the four cytosolic domains (including intracellular loops and the cytoplasmic ends of the transmembrane helices), a sequence profile can be built on the resulting concatenated domains and serve as a discriminator to predict the G protein coupling specificity. Such an approach captures sequence features, if any, spread across 2 or more intracellular loops. In addition, matches to short conserved sequence patterns or motifs (e.g., a single cytosolic domain) may be informative and appropriate in certain cases, but matches to longer sequences (i.e., the four concatenated cytosolic domains) are generally more discriminatory and reliable. As shown in the Examples, three HMMs based on the concatenated cytosolic domains of GPCRs, one each for the G_{i/o}-, G_{q/11}- or Gₛ-class, were constructed. The use of a concatenated sequence to represent each training protein, as opposed to four disparate units, significantly reduces the HMM state space. The HMMs thus constructed were used to predict the G-protein coupling specificity at an accuracy of at least about 95%.

The present invention also features methods for screening drug candidates that modulate the activities of GPCRs. A typical screen method of the present invention includes (1) predicting the G protein coupling specificity of a GPCR of interest using a pattern recognition model of the present invention; and (2) contacting an agent with the GPCR to determine if the agent can modulate the interactions between the GPCR and the predicted G protein, or the signal transduction pathway(s) mediated by the GPCR. Assays suitable for this purpose include, but are not limited to, recombinant cell-based assays, competitive inhibition screens, and biochemical assays.

The recombinant cell-based assays employ expression systems capable of mimicking the in vivo signaling pathway(s) mediated by GPCRs or their coupled G proteins. Expression systems suitable for this purpose include, but are not limited to, yeasts, mammalian cells, insect cells, or amphibian cells. Competitive inhibition screens measure the ability of an agent to replace a bound ligand from a GPCR of interest. The screens can also be used to identify agents capable of preventing ligand binding to the GPCR. Biochemical assays are suitable for screening a large library of agents that may activate or inactivate a signal transduction pathway medicated by a GPCR of interest. An example biochemical assay includes assessments of GPCR coupling to G proteins in the presence or absence of an agent of interest. The selection of appropriate assays or expression systems is a matter of routine design within the level of ordinary skill in the art. An agent thus identified can be any type of molecule, such as a small molecule, a peptide, an oligosaccharide, a lipid, or a combination thereof.

A GPCR modulator identified by the present invention can be formulated into a pharmaceutical composition for treating GPCR-associated diseases, such as cancer, allergies, diabetes, obesity, cardiovascular dysfunction, depression, and a variety of central nervous system disorders. A pharmaceutical composition of the present invention includes a therapeutically effective amount of a GPCR modulator and a pharmaceutically acceptable carrier. Suitable pharmaceutically acceptable carriers include, but are not limited to, solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, that are compatible with pharmaceutical administration. The use of such media and agents for pharmaceutically active substances is well-known in the art. Supplementary agents can also be incorporated into the composition.

A pharmaceutical composition of the present invention can be formulated to be compatible with its intended route of administration. Examples of routes of administration include parenteral, intravenous, intradermal, subcutaneous, oral, inhalative, transdermal, rectal, transmucosal, topical, and systemic administration. In one example, the administration is carried out by an implant.

A pharmaceutical composition of the present invention can be administered to a patient or animal in any desired dosage. A suitable dosage may range, for example, from 5 mg to 100 mg, from 15 mg to 85 mg, from 30 mg to 70 mg, or from 40 mg to 60 mg. Dosages below 5 mg or above 100 mg can also be used. The pharmaceutical composition can be administered in one dose or multiple doses. The doses can be administered at intervals such as once daily, once weekly, or once monthly.

Toxicity and therapeutic efficacy of a GPCR modulator can be determined by standard pharmaceutical procedures in cell culture or experimental animal models. For instance, the LD₅₀ (the dose lethal to 50% of the population) and the ED₅₀ (the dose therapeutically effective in 50% of the population) can be determined. The dose ratio between toxic and therapeutic effects is the therapeutic index, and can be expressed as the ratio LD₅₀/ED₅₀. In many cases, GPCR modulators that exhibit large therapeutic indices are selected.

The data obtained from cell culture assays and animal studies can be used in formulating a range of dosages for use in humans. In one embodiment, the dosage lies within a range of circulating concentrations that exhibit an ED₅₀ with little or no toxicity. The dosage may vary within this range depending upon the dosage form employed and the route of administration utilized.

The dosage regimen for the administration of a GPCR modulator identified by the present invention can be determined by the attending physician based on various factors such as the action of the GPCR modulator, the site of pathology, the severity of disease, the patient's age, sex and diet, the severity of any inflammation, time of administration, and other clinical factors. In one example, systemic or injectable administration is initiated at a dose which is minimally effective, and the dose is increased over a pre-selected time course until a positive effect is observed. Subsequently, incremental increases in dosage are made limiting to levels that produce a corresponding increase in effect while taking into account any adverse affects that may appear.

Progress of a treatment can be monitored by periodic assessment of disease progression. The progress can be monitored, for example, by X-rays, MRI or other imaging modalities, synovial fluid analysis, or clinical examination.

Furthermore, the present invention features systems capable of predicting GPCR-G protein or other protein-ligand interaction specificities. The systems comprise a computer or work station that includes a pattern recognition model of the present invention. The pattern recognition model is a knowledge-restricted model and trained by selected sequence segments of training proteins. In one embodiment, the pattern recognition model is a knowledge-restricted HMM capable of predicting the G protein coupling specificity of an orphan GPCR.

It should be understood that the above-described embodiments and the following examples are given by way of illustration, not limitation. Various changes and modifications within the scope of the invention will become apparent to those skilled in the art from the present description.

### EXAMPLES

### Example 1

### Data Set and HMMs

A set of 102 GPCRs with experimentally determined G protein coupling specificities were selected. The G_{12/13}-class of GPCRs were not included in the study. For simplicity, GPCRs that are known to be promiscuous in coupling were not included in the set. Multiple sequence alignments for the 3 subsets, G_{i/o}-, G_{q/11}-, or Gₛ-classes containing 49, 34 and 19 sequences, respectively, were generated using T-Coffee followed by manual curation of the alignments. Transmembrane (TM) helices of these proteins were predicted using TMHMM (Krogh, et al., J. MOL. BIOL., 305:567-580 (2001)) and in the case of those proteins with fewer than 7 predicted TM helices, TopPred (Claros and Heijne, supra) was used to predict TM helices missed by TMHMM. Blocks of sequences representing the extracellular loops and the predicted TM helices except 2 residues at the cytosolic end of each TM helix were removed from the multiple sequence alignments, leaving behind amino acid residues referred to as cytosolic domains. Excision of TM helix 3 was given special attention so that the E/DRY/F box (Wess, PHARMACOL. THER., 80:231-264 (1998)), when present, is included in i2 regardless of the TM helix prediction. The multiple sequence alignments were further modified by removing sparse columns and columns containing simple repeating patterns. Thus, the multiple sequence alignment of the concatenation of cytosolic domains (i1, i2, i3, and i4, plus the cytosolic ends of the corresponding TM helices) was obtained, and used with the HMMER 2.2 package for building and calibrating HMMs.

For the test set, predicted cytosolic domains were also extracted and concatenated in the same order as the training set. This concatenated sequence was used as query sequence for “hmmpfam” of the HMMER 2.2 package in order to check the match of a GPCR sequence against the set of HMMs.

Two-thirds of the sequences from each subset were randomly chosen as a training set and the remaining one-third were used as test set: No sequence was included in the training set more than once. HMMs for G_{i/o}-, G_{q/11}-, or Gₛ-classes were built using the training set, and the composite test set was used as query sequences. This process of random selection of training set and test set, model-building and model-matching was repeated 100 times resulting in 32 coupling predictions for each protein, on average.

A test GPCR sequence (i.e., concatenation of its predicted cytosolic domains) was matched using “hmmpfam” against the HMMs built for G_{i/o}-, G_{q/11}-, and Gₛ-classes. In the simplistic E-value based method, it is predicted to be specific to the class with the best match (lowest E value) with an E value cutoff of 1.0. A more robust classification based on a discriminant function was carried out as described below.

### Example 2

### Discriminant Analysis

Discriminant analysis was used to assess the rate of misclassifications based on HMM assigned scores. The means of scores Sᵢ, S_{q}, and Sₛ were computed for each sequence. Scores Sᵢ, S_{q}, and Sₛ were HMMER-assigned scores against G_{i/o}-, G_{q/11}-, and Gₛ-specific HMMs, respectively. The data set of mean scores was used in the discriminant function analysis.

Considering a simple example of two classes A₁ and A₂ defined in a space Ω, each class Aᵢ has density function ƒᵢ and prior probability πᵢ. To solve the classification problem is to find a boundary that divides Ω into regions R₁ and R₂ such that if an observation falls in Rᵢ, it will be classified as coming from class Aᵢ. The aim is to minimize the total probability of misclassification
π₂∫_{R}_{₁}ƒ₂dω+π₁∫_{R}_{₂}ƒ₁dω.
By rewriting the above formula as
π₁+∫_{R}_{₁}(π₂ƒ₂−π₁ƒ₁)dω,
the probability is minimized by including in R₁ the points such that π₂ƒ₂<π₁ƒ₁ and excluding from R₁ the points such that π₂ƒ₂>π₁ƒ₁. Continuity of the densities implies that the boundary between R₁ and R₂ is determined by π₁ƒ₁=π₂ƒ₂. When the two densities are multivariate normal with a common within-class covariance matrix, the boundary reduces to a linear discriminant function. When the two densities are multivariate normal with different within-class covariance matrices, it reduces to a quadratic discriminant function. The same conclusions can be generalized to cases with more than two classes.

For discriminant analysis, the data set of 99 sequences with 49, 32 and 18 sequences in G_{i/o}, G_{q/11}- and Gₛ-class, respectively, was considered. Sequences with no replicate data were excluded. The numbers of replicates ranged from 15 to 48. At each of the 2,000 iterations, the data set was split randomly into training set and test set with sizes 66 and 33, respectively. The quadratic discriminant function was developed based on the training set, and applied to the test set. It was assumed that, within each class, the vector of mean scores has a multivariate normal distribution, and each class had its within-class covariance matrix; and, in addition, prior probabilities of the classes were chosen to be equal. SAS version 8.2 (SAS Institute Inc., Cary, N.C.) for the data analysis was employed, and proc discrim for the discriminant analysis in particular.

### Example 3

### Prediction of the Coupling Specificity of GPCRs

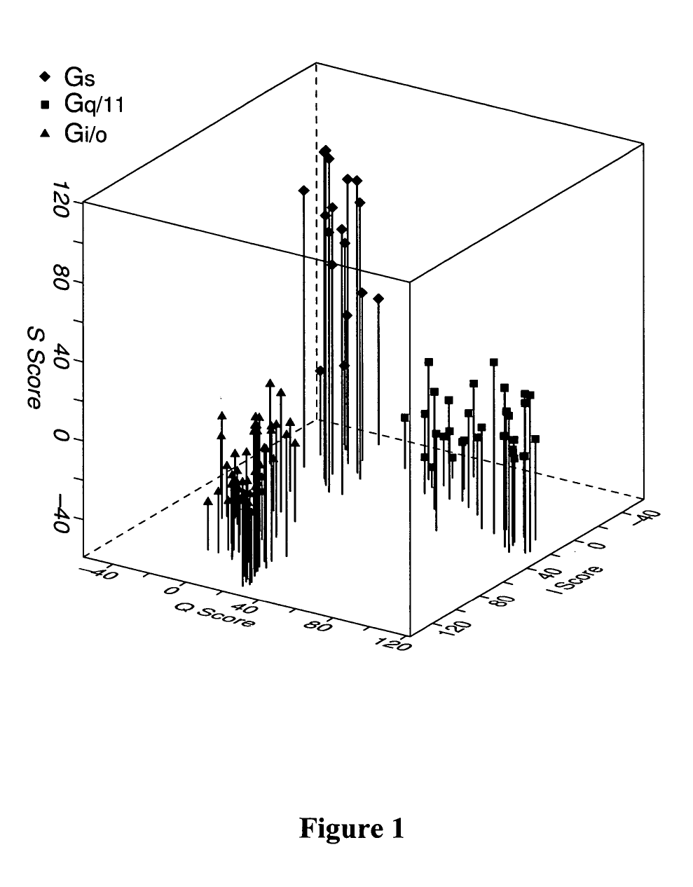

For building and validating the model to predict GPCR-G protein coupling, 49 G_{i/o} class, 34 G_{q/11} class, and 19 Gₛ class of GPCR sequences were used, which had average sequence identities of 26%, 22%, and 24%, respectively, within the cytosolic domain. The most related pair of sequences within these sets had 95%, 82%, and 72% identity and the most unrelated pair had 8%, 4%, and 11% identity within the cytosolic domain of G_{i/o}, G_{q/11}, and Gₛ classes. To avoid bias in segregating training and test sets, training and test sequences were chosen at random and the process was iterated 100 times to dynamically change the contents of the two sets between iterations. Thus in each iteration three HMMs, one for each class, and a test set containing sequences from all three classes, but none included in the training set were created. During the course of these 100 iterations, sequences belonging to the G_{i/o}, G_{q/11}, and Gₛ classes were tested against the HMMs a total of 1,600, 1,100, and 600 times, respectively. A graphical representation of the entire data set generated in the 100 iterations is shown in FIG. 1. It is clear from FIG. 1 that all the G_{i,o} coupling GPCRs have high scores against G_{i/o}-specific HMMs (the “I Score”), but low scores against G_{q/11}- (the “Q score”) and Gₛ-specific (the “S Score”) HMMs. Similarly, the G_{q/11}- and Gₛ-coupling GPCRs have high scores against their respective class-specific HMMs and low scores against HMMs specific for a different class.

The raw predictions are also presented in Tables 1, 2, and 3. Knowledge-restricted HMM has the best result in the case of G_{i/o}-coupled GPCR sequences. In this class only a single case of wrong prediction was reported by EDG2. For the G_{q}-coupled GPCRs, there were only two GPCRs that were misclassified at least once—namely, MGR1 and MGR5. Finally, for the Gₛ family, there were three possible misclassifications—namely, FSHR, PI2R, and V2R. Thus, even by taking simply a single prediction, the chances of misclassification were relatively small. In order to estimate the robustness with which the classification between various classes is made, the discriminant analysis described in Example 2 was conducted. 136 misclassifications were identified, equivalent to an error rate of 0.0021.

In order to evaluate the benefits of knowledge-restricted HMMs, HMMs were created using the multiple sequence alignments of full-length sequences and then tested by full-length query sequences. In contrast to the high accuracy rate of the knowledge-restricted HMMs, the predictions made by full-length HMMs and full-length query sequences were error prone.

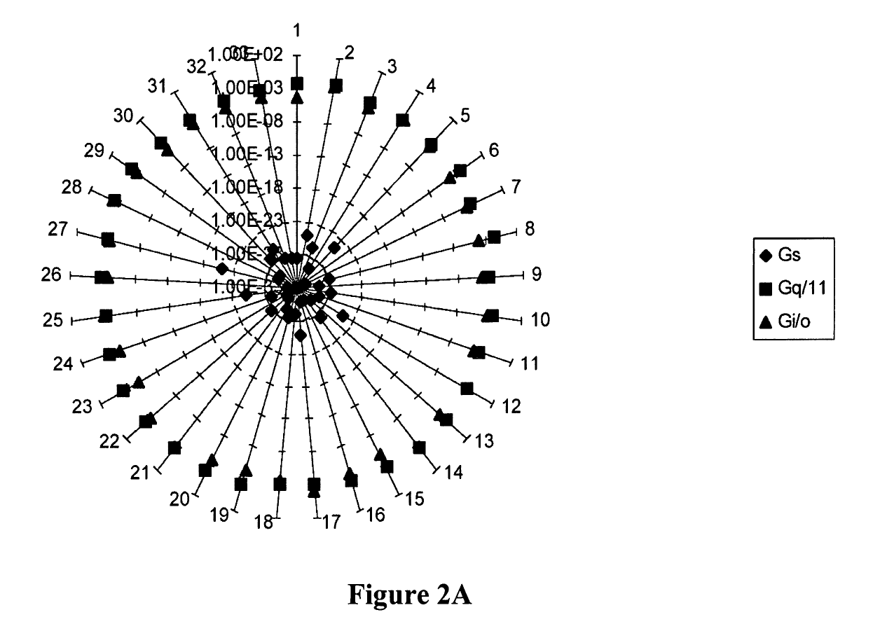

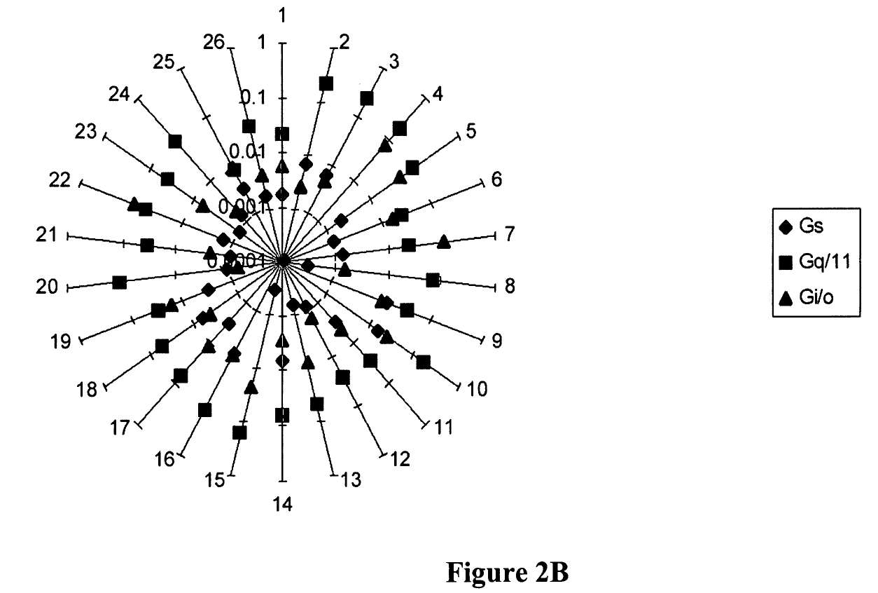

FIGS. 2A and 2B are radar plots showing the E-values obtained for melanocortin 3 receptor (MC3R) and follicle stimulating hormone receptor (FSHR), respectively, against the Gₛ-, G_{i/o}-, and G_{q/11}-specific HMMs. It was noticed from FIG. 2A that there was a unanimous verdict regarding the coupling specificity of MC3R with extremely low E-values against the Gₛ-specific HMMs. Also, there is a significant difference between the E-values obtained against the Gₛ-specific HMMs and those against the G_{i/o} and G_{q/11}-specific HMMs. In the case of FSHR, the verdict was not unanimous, though a vast majority of the models predicted FSHR to be Gₛ-coupling (FIG. 2B). As depicted in FIG. 2B, the E-values of FSHR against different Gₛ-, G_{q/11}- and G_{i/o}-specific HMMs were slightly overlapping and not drastically different between classes. These two plots represent the kind of variation observed in the attempt to predict G protein coupling.

Of the 1,600 predictions based on E-value, there was one wrong prediction in the G_{i/o} class of proteins (Table 1). The lysophosphatidic acid receptor (EDG2, SwissProt: Q92633) was tested 24 times against different HMMs and was misclassified as Gₛ coupling once and correctly classified as G_{i/o} coupling 23 times. The discriminant function also misclassified EDG2 twice in 631 attempts.

TABLE 1
List of G_{i/o}-Coupling GPCRs and Their Classification Based on Knowledge-Restricted HMMs^{$}

| | | No. of times included | No. of times classified as | | | |
|---|---|---|---|---|---|---|
| Accession** | SwissProt Locus | in test set | G_{i/o}-class | G_{q/11}-class | Gₛ-class | Failed* |
| P08172 | ACM2_HUMAN | 28 (675) | 28 (675) | 0 (0) | 0 (0) | 0 (0) |
| P08173 | ACM4_HUMAN | 38 (681) | 38 (681) | 0 (0) | 0 (0) | 0 (0) |
| P30542 | AA1R_HUMAN | 26 (678) | 26 (678) | 0 (0) | 0 (0) | 0 (0) |
| P33765 | AA3R_HUMAN | 33 (657) | 33 (657) | 0 (0) | 0 (0) | 0 (0) |
| P08913 | A2AA_HUMAN | 28 (665) | 28 (665) | 0 (0) | 0 (0) | 0 (0) |
| P18089 | A2AB_HUMAN | 32 (504) | 32 (504) | 0 (0) | 0 (0) | 0 (0) |
| P18825 | A2AC_HUMAN | 41 (606) | 41 (606) | 0 (0) | 0 (0) | 0 (0) |
| P21554 | CB1R_HUMAN | 30 (703) | 30 (703) | 0 (0) | 0 (0) | 0 (0) |
| P34972 | CB2R_HUMAN | 33 (667) | 33 (667) | 0 (0) | 0 (0) | 0 (0) |
| P32246 | CKR1_HUMAN | 42 (648) | 42 (648) | 0 (0) | 0 (0) | 0 (0) |
| P41597 | CKR2_HUMAN | 40 (669) | 40 (669) | 0 (0) | 0 (0) | 0 (0) |
| P51677 | CKR3_HUMAN | 32 (655) | 32 (655) | 0 (0) | 0 (0) | 0 (0) |
| P51679 | CKR4_HUMAN | 31 (660) | 31 (660) | 0 (0) | 0 (0) | 0 (0) |
| P51684 | CKR6_HUMAN | 30 (661) | 30 (661) | 0 (0) | 0 (0) | 0 (0) |
| P32248 | CKR7_HUMAN | 36 (654) | 36 (654) | 0 (0) | 0 (0) | 0 (0) |
| P51685 | CKR8_HUMAN | 35 (646) | 35 (646) | 0 (0) | 0 (0) | 0 (0) |
| P51686 | CKR9_HUMAN | 30 (659) | 30 (659) | 0 (0) | 0 (0) | 0 (0) |
| P46092 | CKRA_HUMAN | 34 (658) | 34 (658) | 0 (0) | 0 (0) | 0 (0) |
| P25024 | IL8A_HUMAN | 38 (668) | 38 (668) | 0 (0) | 0 (0) | 0 (0) |
| P25025 | IL8B_HUMAN | 31 (677) | 31 (677) | 0 (0) | 0 (0) | 0 (0) |
| P49682 | CCR3_HUMAN | 44 (669) | 44 (669) | 0 (0) | 0 (0) | 0 (0) |
| P30991 | CCR4_HUMAN | 36 (668) | 36 (668) | 0 (0) | 0 (0) | 0 (0) |
| P32302 | CCR5_HUMAN | 32 (657) | 32 (657) | 0 (0) | 0 (0) | 0 (0) |
| P49238 | C3X1_HUMAN | 33 (736) | 33 (736) | 0 (0) | 0 (0) | 0 (0) |
| P46094 | CXC1_HUMAN | 32 (661) | 32 (661) | 0 (0) | 0 (0) | 0 (0) |
| P14416 | D2DR_HUMAN | 28 (663) | 28 (663) | 0 (0) | 0 (0) | 0 (0) |
| P35462 | D3DR_HUMAN | 39 (661) | 39 (661) | 0 (0) | 0 (0) | 0 (0) |
| P21917 | D4DR_HUMAN | 28 (626) | 28 (626) | 0 (0) | 0 (0) | 0 (0) |
| O60755 | GALT_HUMAN | 40 (663) | 40 (663) | 0 (0) | 0 (0) | 0 (0) |
| P28222 | 5H1B_HUMAN | 31 (578) | 31 (578) | 0 (0) | 0 (0) | 0 (0) |
| P28221 | 5H1D_HUMAN | 34 (714) | 34 (714) | 0 (0) | 0 (0) | 0 (0) |
| P28566 | 5H1E_HUMAN | 28 (757) | 28 (757) | 0 (0) | 0 (0) | 0 (0) |
| P30939 | 5H1F_HUMAN | 26 (724) | 26 (724) | 0 (0) | 0 (0) | 0 (0) |
| Q92633 | EDG2_HUMAN | 24 (631) | 23 (629) | 0 (0) | 1 (2) | 0 (0) |
| P48039 | ML1A_HUMAN | 27 (674) | 27 (674) | 0 (0) | 0 (0) | 0 (0) |
| P49286 | ML1B_HUMAN | 27 (705) | 27 (705) | 0 (0) | 0 (0) | 0 (0) |
| P25929 | NY1R_HUMAN | 36 (701) | 36 (701) | 0 (0) | 0 (0) | 0 (0) |
| P49146 | NY2R_HUMAN | 32 (686) | 32 (686) | 0 (0) | 0 (0) | 0 (0) |
| P50391 | NY4R_HUMAN | 34 (673) | 34 (673) | 0 (0) | 0 (0) | 0 (0) |
| Q15761 | NY5R_HUMAN | 31 (654) | 31 (654) | 0 (0) | 0 (0) | 0 (0) |
| P41143 | OPRD_HUMAN | 30 (664) | 30 (664) | 0 (0) | 0 (0) | 0 (0) |
| P41145 | OPRK_HUMAN | 29 (669) | 29 (669) | 0 (0) | 0 (0) | 0 (0) |
| P35372 | OPRM_HUMAN | 31 (673) | 31 (673) | 0 (0) | 0 (0) | 0 (0) |
| P41146 | OPRX_HUMAN | 35 (664) | 35 (664) | 0 (0) | 0 (0) | 0 (0) |
| P30872 | SSR1_HUMAN | 32 (558) | 32 (558) | 0 (0) | 0 (0) | 0 (0) |
| P30874 | SSR2_HUMAN | 38 (695) | 38 (695) | 0 (0) | 0 (0) | 0 (0) |
| P32745 | SSR3_HUMAN | 30 (822) | 30 (822) | 0 (0) | 0 (0) | 0 (0) |
| P31391 | SSR4_HUMAN | 39 (805) | 39 (805) | 0 (0) | 0 (0) | 0 (0) |
| P35346 | SSR5_HUMAN | 26 (682) | 26 (682) | 0 (0) | 0 (0) | 0 (0) |

*E-value > 1.00 for the best match.
**Accession numbers are from SwissProt/TREMBL database.
^{$}In columns 3-7 numbers inside the parenthesis were obtained from the discriminant analysis.

As shown in Table 2, there were 12 misclassifications in a total of 1,100 predictions based on E-value for the G_{q/11} class of receptors. All 12 misclassifications were either for metabotropic glutamate receptor 1 precursor (MGR1, SwissProt: Q13255) or metabotropic glutamate receptor 5 precursor (MGR5, SwissProt: P41594). The MGR 1 precursor was included 27 times in the test set; it was classified as G_{i/o} coupling 3 times, 7 times it was not matched against any 3 models at E-value <1.0 and the remaining 17 times it was correctly classified. Of the 26 times MGR5 was tested, correct classification was made 15 times, but 3 times it was classified as G_{i/o} coupling, 1 time as Gₛ coupling and 7 times it was not matched against any 3 models at E-value <1.0. MGR1 and MGR5 were not included in the discriminant analysis because of insufficient data points.

Of the 600 predictions based on E-value for the Gₛ class of proteins, 13 were wrong; all mistakes were limited to 3 sequences (Table 3)—namely, FSHR, V2R, and PI2R. The follicle stimulating hormone receptor precursor (FSHR, SwissProt: P23945) was correctly classified 20 times, but wrongly classified as G_{i/o} coupling on 6 occasions (Table 3 and FIG. 1B). As expected, the discriminant function also misclassified FSHR in 115 of the 665 attempts. Similarly, based on E-value vasopressin V2 receptor (V2R, SwissProt: P30518) was correctly classified 28 times, but wrongly classified as G_{q/11} coupling on 6 occasions. For V2R, the error rate in the discriminant analysis was 15 out of 692 attempts. The prostacyclin receptor (PI2R, SwissProt: P43119) was correctly classified on 27 of the 28 attempts and wrongly placed into the G_{q/11} class on one occasion. The prostaglandin E2 receptor (PE24, SwissProt: P35408) and PI2R were misclassified by the discriminant function at an error rate of 1 out of 662 and 2 out of 681, respectively. Prostaglandin D2 receptor (PD2R, SwissProt: Q13258) was not included in the discriminant analysis because of insufficient data points in G_{i/o} and G_{q/11} scores.

TABLE 2
List of G_{q/11}-Coupling GPCRs and Their Classification Based on Knowledge-Restricted HMMs^{$}

| | | No. of times | No. of times classified as | | | |
|---|---|---|---|---|---|---|
| Accession** | SwissProt Locus | included in test set | G_{i/o}-class | G_{q/11}-class | Gₛ-class | Failed* |
| P11229 | ACM1_HUMAN | 48 (624) | 0 (0) | 48 (624) | 0 (0) | 0 (0) |
| P20309 | ACM3_HUMAN | 31 (649) | 0 (0) | 31 (649) | 0 (0) | 0 (0) |
| P08912 | ACM5_HUMAN | 33 (656) | 0 (0) | 33 (656) | 0 (0) | 0 (0) |
| P35348 | A1AA_HUMAN | 31 (655) | 0 (0) | 31 (655) | 0 (0) | 0 (0) |
| P35368 | A1AB_HUMAN | 32 (660) | 0 (0) | 32 (660) | 0 (0) | 0 (0) |
| P25100 | A1AD_HUMAN | 38 (665) | 0 (0) | 38 (665) | 0 (0) | 0 (0) |
| P28336 | NMBR_HUMAN | 26 (679) | 0 (0) | 26 (679) | 0 (0) | 0 (0) |
| P30550 | GRPR_HUMAN | 38 (669) | 0 (0) | 38 (669) | 0 (0) | 0 (0) |
| P32247 | BRS3_HUMAN | 37 (660) | 0 (0) | 37 (660) | 0 (0) | 0 (0) |
| P46663 | BRB1_HUMAN | 15 (683) | 0 (0) | 15 (683) | 0 (0) | 0 (0) |
| P30411 | BRB2_HUMAN | 37 (663) | 0 (0) | 37 (663) | 0 (0) | 0 (0) |
| P32239 | GASR_HUMAN | 26 (672) | 0 (0) | 26 (672) | 0 (0) | 0 (0) |
| Q13255 | MGR1_HUMAN | 27 ( ) | 3 ( ) | 17 ( ) | 0 ( ) | 7 ( ) |
| P41594 | MGR5_HUMAN | 26 ( ) | 3 ( ) | 15 ( ) | 1 ( ) | 7 ( ) |
| P35367 | HH1R_HUMAN | 29 (666) | 0 (0) | 29 (666) | 0 (0) | 0 (0) |
| P28223 | 5H2A_HUMAN | 31 (682) | 0 (0) | 31 (682) | 0 (0) | 0 (0) |
| P41595 | 5H2B_HUMAN | 32 (677) | 0 (0) | 32 (677) | 0 (0) | 0 (0) |
| P28335 | 5H2C_HUMAN | 36 (654) | 0 (0) | 36 (654) | 0 (0) | 0 (0) |
| Q9Y271 | CLT1_HUMAN | 33 (672) | 0 (0) | 33 (672) | 0 (0) | 0 (0) |
| P30989 | NTR1_HUMAN | 32 (682) | 0 (0) | 32 (682) | 0 (0) | 0 (0) |
| O95665 | NTR2_HUMAN | 37 (667) | 0 (0) | 37 (667) | 0 (0) | 0 (0) |
| P47900 | P2YR_HUMAN | 33 (676) | 0 (0) | 33 (676) | 0 (0) | 0 (0) |
| P41231 | P2UR_HUMAN | 38 (672) | 0 (0) | 38 (672) | 0 (0) | 0 (0) |
| P51582 | P2Y4_HUMAN | 31 (655) | 0 (0) | 31 (655) | 0 (0) | 0 (0) |
| Q15077 | P2Y6_HUMAN | 35 (666) | 0 (0) | 35 (666) | 0 (0) | 0 (0) |
| P43088 | PF2R_HUMAN | 22 (683) | 0 (0) | 22 (683) | 0 (0) | 0 (0) |
| P21731 | TA2R_HUMAN | 38 (673) | 0 (0) | 38 (673) | 0 (0) | 0 (0) |
| P34995 | PE21_HUMAN | 38 (672) | 0 (0) | 38 (672) | 0 (0) | 0 (0) |
| P25103 | NK1R_HUMAN | 32 (688) | 0 (0) | 32 (688) | 0 (0) | 0 (0) |
| P21452 | NK2R_HUMAN | 36 (681) | 0 (0) | 36 (681) | 0 (0) | 0 (0) |
| P29371 | NK3R_HUMAN | 26 (656) | 0 (0) | 26 (656) | 0 (0) | 0 (0) |
| Q9UKP6 | UR2R_HUMAN | 32 (654) | 0 (0) | 32 (654) | 0 (0) | 0 (0) |
| P37288 | V1AR_HUMAN | 26 (660) | 0 (0) | 26 (660) | 0 (0) | 0 (0) |
| P47901 | V1BR_HUMAN | 38 (658) | 0 (0) | 38 (658) | 0 (0) | 0 (0) |

*E-value > 1.00 for the best match.
**Accession numbers are from SwissProt/TREMBL database.
^{$}In columns 3-7 numbers inside the parenthesis were obtained from the discriminant analysis.

TABLE 3
List of Gₛ-Coupling GPCRs and Their Classification Based on Knowledge-Restricted HMMs^{$}

| | | No. of times | No. of times classified as | | | |
|---|---|---|---|---|---|---|
| Accession** | SwissProt Locus | included in test set | G_{i/o}-class | G_{q/11}-class | Gₛ-class | Failed* |
| P29274 | AA2A_HUMAN | 35 (652) | 0 (0) | 0 (0) | 35 (652) | 0 (0) |
| P29275 | AA2B_HUMAN | 31 (660) | 0 (0) | 0 (0) | 31 (660) | 0 (0) |
| P08588 | B1AR_HUMAN | 34 (661) | 0 (0) | 0 (0) | 34 (661) | 0 (0) |
| P07550 | B2AR_HUMAN | 38 (647) | 0 (0) | 0 (0) | 38 (647) | 0 (0) |
| P21728 | DADR_HUMAN | 20 (659) | 0 (0) | 0 (0) | 20 (659) | 0 (0) |
| P21918 | DBDR_HUMAN | 32 (654) | 0 (0) | 0 (0) | 32 (654) | 0 (0) |
| P23945 | FSHR_HUMAN | 26 (665) | 6 (113) | 0 (2) | 20 (550) | 0 (0) |
| P50406 | 5H6_HUMAN | 29 (634) | 0 (0) | 0 (0) | 29 (634) | 0 (0) |
| P34969 | 5H7_HUMAN | 37 (642) | 0 (0) | 0 (1) | 37 (641) | 0 (0) |
| Q01726 | MSHR_HUMAN | 36 (647) | 0 (0) | 0 (0) | 36 (647) | 0 (0) |
| Q01718 | ACTR_HUMAN | 26 (656) | 0 (0) | 0 (0) | 26 (656) | 0 (0) |
| P41968 | MC3R_HUMAN | 33 (645) | 0 (0) | 0 (0) | 33 (645) | 0 (0) |
| P32245 | MC4R_HUMAN | 25 (676) | 0 (0) | 0 (0) | 25 (676) | 0 (0) |
| P33032 | MC5R_HUMAN | 31 (649) | 0 (0) | 0 (0) | 31 (649) | 0 (0) |
| Q13258 | PD2R_HUMAN | 35 ( ) | 0 ( ) | 0 ( ) | 35 ( ) | 0 ( ) |
| P43119 | PI2R_HUMAN | 28 (681) | 0 (2) | 1 (0) | 27 (679) | 0 (0) |
| P43116 | PE22_HUMAN | 37 (665) | 0 (0) | 0 (0) | 37 (665) | 0 (0) |
| P35408 | PE24_HUMAN | 33 (662) | 0 (1) | 0 (0) | 33 (661) | 0 (0) |
| P30518 | V2R_HUMAN | 34 (692) | 0 (15) | 6 (0) | 28 (677) | 0 (0) |

*E-value > 1.00 for the best match.
**Accession numbers are from SwissProt/TREMBL database.
^{$}In columns 3-7 numbers inside the parenthesis were obtained from the discriminant analysis.

The assumptions of this Example for the GPCR-G protein coupling prediction are the following: (1) intracellular loops and the cytosolic ends of the transmembrane segments, together referred to as the cytosolic domain, may contribute to the specificity of GPCR-G protein coupling; (2) although interrupted by TM sequences and/or extracellular loops in the primary structure of the GPCRs, the four intracellular segments (i1, i2, i3 and i4) treated as a contiguous sequence of amino acids may provide a reasonable framework for building a hidden Markov model that captures the features of the coupling domain; (3) when determining the match between a model and the sequence of a GPCR, the cytosolic domain may be extracted and used as query instead of the full sequence. The premise that sequence similarity can predict G-protein coupling selectivity appears to be inconsistent with certain arguments articulated by Wong, NEUROSIGNALS, 12:1-12 (2003). According to Wong's hypothesis, G protein selectivity is defined by the conformation of the intracellular region of GPCRs and this conformation is regulated by the interaction between several intracellular regions. Further, G protein coupling selectivity was considered a result of a combination of a general “activation domain” and a specific “selectivity domain.” See Wong, supra. The inability to find a consensus G protein-coupling motif amongst GPCRs may be because the “consensus motif” is comprised of sequences from two or more intracellular regions, and many previous attempts at identifying such motifs considered the four intracellular regions in isolation.

In order to classify the proteins into G_{i,o}, G_{q/11} and Gₛ classes, two approaches were followed: (1) a simplistic, best E-value based approach; and (2) one based on a discriminant function that uses the HMM-assigned scores rather than the E-values. Both the methods gave similar results, as expected, because E-values are derived from the scores. It is evident from the data presented in Tables 1, 2 and 3 that the sequence of the concatenated cytosolic domains can provide enough signal to correctly classify GPCRs according to their coupling preference. The error rate of the prediction scheme over 100 iterations as described in this Example was less than 1.00%. When full-length sequences were used as training and test sequences, instead of the concatenated cytosolic domains, the error rates were 6%, 27% and 41% for the G_{i/o}, G_{q/11}- and Gₛ-classes, respectively, with an overall error rate of 19%. This high error rate observed when full-length sequences were used underscores the advantage of applying biological intuition, in this case using only the presumed relevant fragments, in the development of improved computational tools for biology.

Computational tools such as HMMs and artificial neural nets can be built for finding patterns in data. While they generally perform creditably, the models often deliberately ignore well-known patterns in the data with the assumption that the pattern detection tool will find it anyway. In the case of protein sequences, different patterns may exist at different positions for entirely different reasons. For a GPCR, the transmembrane segments are hydrophobic, the extracellular domains and transmembrane segments hold patterns for non-G protein ligand specificity and the intracellular domains for G-protein specificity. Since hydrophobicity and non-G protein ligand specificity are not related to G-protein specificity, including those sequences in the HMM might lead to dilution of the pattern or to a weaker HMM. The high error rate noted from the use of full length sequences for model building and testing supports this analysis.

The GPCR-G protein coupling prediction strategy presented in this Example showed ambiguity in the case of a few receptors. Of the sequences that were not unanimously segregated by the hidden Markov models, EDG2 was the lone member of G_{i/o}-class (Table 1). There are indications that EDG2 is capable of coupling to G_{i/o}, G_{q/11} and G_{12/13}. Table 2 reveals that coupling prediction of two proteins of the G_{q/11}-class, MGR1 and MGR5, were ambiguous. Experimental evidence exists for Gₛ-coupling and G_{i/o}-coupling by MGR1. MGR1-G_{i/o} coupling was predicted by 3 out of 27 models, but 7 of the 27 models did not yield a prediction for the same receptor because of E-values higher than the threshold used in this study. The coupling prediction for MGR5 was also not unanimous although the majority of the models predicted it to be of the G_{q/11}-class. The Gₛ-coupling FSHR was predicted to belong to the G_{i/o}-class by 6 of the 26 models (Table 3, FIG. 2b). FSHR coupling to both adenylyl cyclase and phospholipase C cascades in CHO cells has been suggested, but in contrast to the predictions by the knowledge-restricted HMMs, there is as yet no evidence for a G_{i/o}-mediated response. The Gs-coupling prostacyclin receptor PI2R was predicted to belong to the G_{q/11}-class by one of the 28 models (Table 3). This receptor was suggested to couple to G_{q/11} in addition to Gₛ. V2 vasopressin receptor V2R is another Gs-coupling protein that was predicted to couple to G_{q/11} by 6 of the 34 models. Single amino acid substitution (M145L) in the second intracellular loop of V2R was sufficient to show substantial coupling to G_{q5}. Other members of the vasopressin/oxytocin receptor family selectively couple to G_{q/11} and have a leucine at the position corresponding to this methionine (M145).

Currently promiscuity in GPCR-G protein coupling is well established for 18 receptors. It is likely that more receptors will join this promiscuous group as more cell-types, physiological conditions and receptors are studied. A Bayesian classification scheme of G-protein coupling predicted promiscuity for 35 of the 55 receptors included in the validation set. As mentioned previously, none of the 102 receptors selected in the present study are considered to be promiscuous in G coupling. However a few models, albeit a small fraction, indicated promiscuity for 6 of the 102 receptors and 4 of this 6 receptors have been suggested or shown to be promiscuous. An example is shown in FIG. 2b that suggests that FSHR might be promiscuous in G-protein coupling. Ambiguous predictions may be the starting points for further experiments exploring alternative G protein coupling and downstream signal processing events rather than being dismissed as in silico artifacts.

Among the factors that might influence GPCR-G protein coupling, but not considered by the prediction scheme described in this paper, is post-translational modification of the receptor.

Relatively small number of sequences of the G_{q/11}- and Gₛ-classes of receptors are available for model building; this may have an adverse impact on the prediction ability for these classes. The method described in this Example has the highest error rate for the Gₛ-class for which the training set was the smallest and the lowest error rate for the G_{i/o}-class for which the training set was the largest. The lower error rate in the G_{i/o}-class when compared to the error rates in G_{q/11}- and Gₛ-classes might represent a reflection of the size of the training set and not because of a more discriminant or restrictive profile of the G_{i/o}-class that enables predictions at low rate. Sensitivity and selectivity of the prediction method of this Example might be improved with the availability of a larger training set. Thus, as more data becomes available (promiscuity as well as for all specificities), improved knowledge-restricted HMMs with better prediction performance may be constructed according to the present invention.

In a number of situations in computational biology, it is expected that knowledge restriction of HMMs or other pattern recognition tools may give rich rewards. Deorphaning a receptor is a significant milestone in understanding the GPCR. It is possible that, when a number of GPCRs that bind to similar extracellular ligands are known, other GPCRs of similar specificities can be identified using a knowledge-restricted HMM using only the extracellular domains. Another example is MHC-peptide binding, where only the binding groove sequence is expected to have any significant impact on peptide selectivity of an MHC. It is possible to build an HMM of just the MHC peptide binding groove in order to get a relatively compact model of peptide binding specificity.

The principle of knowledge restriction in building biological models may be adapted to methods other than HMMs. For example, principal component analysis (PCA), partial least squares analysis (PLS), and support vector machines (SVMs) can be similarly employed for classification of GPCRs.

The foregoing description of the invention provides illustration and description, but is not intended to be exhaustive or to limit the invention to the precise one disclosed. Modifications and variations consistent with the above teachings are possible or may be acquired from practice of the invention. Thus, it is noted that the scope of the invention is defined by the claims and their equivalents.

What is claimed is:

1. A method for evaluating G protein coupling specificity of a G protein-coupled receptor (GPCR) of interest, said method comprising:
training a pattern recognition model with a plurality of training sequences, said training sequences being derived from a group of training GPCRs which are capable of interacting with a specified class of G proteins, each said training sequence comprising a concatenation of two or more non-contiguous sequence segments of a training GPCR selected from said group, and each said non-contiguous sequence segment comprising an intracellular sequence of said training GPCR; and
querying the trained model with a query sequence which comprises a concatenation of two or more non-contiguous sequence segments of the GPCR of interest, each said non-contiguous sequence segment of the GPCR of interest comprising an intracellular sequence of the GPCR of interest,
wherein a match or no-match of said query sequence to the trained model is indicative of whether or not the GPCR of interest is capable of interacting with said specified class of G proteins.

2. The method of claim 1, wherein each said training sequence comprises a concatenation of two or more cytosolic domains of a training GPCR selected from said group, and said query sequence comprises a concatenation of two or more cytosolic domains of the GPCR of interest.

3. The method of claim 1, wherein each said training sequence comprises a concatenation of four cytosolic domains of a training GPCR selected from said group, and said query sequence comprises a concatenation of four cytosolic domains of the GPCR of interest.

4. The method of claim 3, wherein said pattern recognition model is a hidden Markov model.

5. The method of claim 4, wherein said querying generates an E-value or an HMMER score which indicates a match or no-match of said query sequence to the trained model.

6. The method of claim 5, wherein said specified class of G proteins is selected from the group consisting of G_{i/o} class, G_{q/11} class, Gₛ class, and G_{12/13} class.

7. The method of claim 5, wherein the GPCR of interest is an orphan GPCR.

8. The method of claim 5, wherein said group of training GPCRs and the GPCR of interest are alignable in a multiple sequence alignment, and the non-contiguous sequence segments of said training GPCR are alignable to the non-contiguous sequence segments of the GPCR of interest in said multiple sequence alignment.

9. The method of claim 8, wherein said multiple sequence alignment is produced by a T-Coffee program.

10. A method for identifying modulators of interactions between a GPCR of interest and G proteins, said method comprising:
identifying a class of G proteins capable of interacting with the GPCR of interest according to the method of claim 1; and
monitoring an interaction between the GPCR of interest and a G protein selected from said class in the presence or absence of an agent,
wherein a change in said interaction in the presence of said agent, as compared to in the absence of said agent, indicates that said agent modulates said interaction between the GPCR of interest and said G protein.

11. The method of claim 10, wherein said agent is an agonist or antagonist of the GPCR of interest.

12. The method of claim 10, wherein the GPCR of interest is an orphan GPCR.

13. A method for modulating a signal transduction pathway mediated by a GPCR of interest, comprising:
identifying a class of G proteins capable of interacting with the GPCR of interest according to the method of claim 1;
providing an agent capable of modulating a signal transduction pathway mediated by a G protein selected from said class; and
introducing said agent into a cell which comprises the GPCR of interest and said G protein.

14. A method for building a pattern recognition model for evaluating G protein coupling specificity of GPCRs, comprising:
preparing training sequences from a plurality of GPCRs which have a specified G protein coupling specificity, each said training sequence comprising a concatenation of two or more non-contiguous sequence segments of a GPCR selected from said plurality of GPCRs, and each said non-contiguous sequence segment comprising an intracellular sequence of said GPCR; and
training said pattern recognition model with said training sequences.

15. The method of claim 14, wherein said pattern recognition model is a hidden Markov model.

16. The method of claim 14, wherein each said training sequence comprises a concatenation of four cytosolic domains of a GPCR selected from said plurality of GPCRs.

17. A system comprising a pattern recognition model trained by a plurality of training sequences, wherein each said training sequence comprises a concatenation of two or more non-contiguous sequence segments of a GPCR which has a specified G protein coupling specificity, and each said non-contiguous sequence segment comprises an intracellular sequence of said GPCR.

18. The system of claim 17, wherein said pattern recognition model is a hidden Markov model, and each said training sequence comprises a concatenation of four cytosolic domains of a GPCR.

19. A method for evaluating ligand coupling specificity of a protein of interest, comprising:
training a pattern recognition model with a plurality of training sequences, said training sequences being derived from a group of training proteins which have a specified ligand coupling specificity, each said training sequence comprising a concatenation of two or more non-contiguous sequence segments of a training protein selected from said group; and
querying the trained model with a query sequence which comprises a concatenation of two or more non-contiguous sequence segments of the protein of interest,
wherein a match or no-match of said query sequence to the trained model is indicative of whether or not the protein of interest has said specified ligand coupling specificity.

20. The method of claim 19, wherein said pattern recognition model is a hidden Markov model.
